(19) United States
(12) Patent Application Publication
(10) Pub. No.: US 2014/0277553 A1
(43) Pub. Date: Sep. 18, 2014

(54) Ulnar Head Replacement Implant System

(71) Applicant: Ascension Orthopedics, Inc., Austin, TX (US)

(72) Inventors: Jerome J. Klawitter, Austin, TX (US); Monti R. Gourley, Austin, TX (US)

(73) Assignee: Ascension Orthopedics, Inc., Austin, TX (US)

(21) Appl. No.: 14/208,735

(22) Filed: Mar. 13, 2014

Related U.S. Application Data
(60) Provisional application No. 61/783,553, filed on Mar. 14, 2013.

(51) Int. Cl. A61F 2/42

(52) U.S. Cl. CPC A61F 2/4261
USPC 623/21.12

(57) ABSTRACT

Embodiments of the invention may provide a stem and a head and a liner that can be assembled together to form an implant, such as may be used in repair of the distal ulnoradial joint. A given head and liner and stem may be assembled to each other in more than one configuration, providing more than one possible external shape for a given set of stem and head and liner. The head may be non-symmetric about its midplane and may be open at both ends. A kit may contain more than one head assemblable to a given stem with an intermediate liner, with the various heads providing various different external shapes of the device.

### FIELD OF THE INVENTION

Embodiments of the invention pertain to surgery for the repair of skeletal joints.

### BACKGROUND OF THE INVENTION

In the human arm, the side of the distal ulna articulates with the side of the distal radius, thereby allowing the hand pronation and supination. Disease or trauma sometimes requires replacement of the head of the distal ulna. Various materials and designs for replacement parts are known. However, there remains a need for better designs that will provide the surgeon with appropriate options during surgery while not requiring a large quantity and inventory of parts.

Referring now to FIG. 1, there are illustrated three different spatial relationships that are possible for the articulation between the distal ulna and distal radius, i.e., the distal radioulnar joint (DRUJ). These various spatial relationships may be designated Type I joints, Type II joints, and Type III joints. FIG. 1 illustrates a centerline of the ulna, and illustrates a line that is tangent to the articulation surface at which the distal ulna articulates with the distal radius. There is further illustrated an angle between those other two lines, which may be referred to as the DRUJ angle.

In a Type I joint, the surface of the articulation is substantially parallel to the ulnar centerline. In Type II joints, the surface of the articulation is inclined to the ulnar centerline in one direction. In Type III joints, the surface of the articulation is inclined to the ulnar centerline in the opposite direction.

All three of these spatial relationships occur in normal human anatomy, in different individuals. Based on a study of fifty cadaveric wrist specimens. Tolat et al (Tolat A R, Stanley J K, Trail I A, A cadaveric study of the anatomy of and stability of the distal radioulnar joint in the coronal and transverse planes, J Hand Surg. Vol. 21B, No. 5, October 1996) report the distribution of the three types as follows; Type I, 55%; Type II, 33% and Type III, 12%. In the Type I wrist the DRUJ angle was essentially zero. The distribution of the DRUJ angles for Type II and Type III wrists, as reported by Tolat, et al., are shown in Table 1. Type II DRUJ angles were predominantly in the 10 degree to 20 degree range whereas the Type III DRUJ angles were mainly in the 15 degree to 20 degree range.

TABLE 1
Distribution of the DRUJ angles for Type I, Type II and Type III wrists
DRUJ Types and DRUJ Angles as Reported by Tolat et. al.

| DRUJ Joint | | DRUJ Angle Range, Degrees | | | |
|---|---|---|---|---|---|
| Type | Occurrence | 0 | 10 to 15 | 16 to 20 | 21 to 24 |
| Type I | 55% | 100% | 0% | 0% | 0% |
| Type II | 33% | 0% | 81% | 6% | 13% |
| Type III | 12% | 0% | 17% | 67% | 17% |

TABLE 2
Occurrence of DRUJ angles based on all fifty of the Tolat
et. al. cadaver specimens and representing each range
of DRUJ angles by its average

DRUJ Average Angle Range, Degrees

| DRUJ Type | 0 | 12.5 | 18 | 22.5 |
|---|---|---|---|---|
| Type I | 55% | 0% | 0% | 0% |
| Type II | 0% | 27% | 2% | 4% |
| Type III | 0% | 2% | 8% | 2% |
| Total | 55% | 29% | 10% | 6% |

Tolat et. al. report DRUJ angles ranges for all Types (I, II and III) as groupings of 0 degrees, and ranges of 10 to 15 degrees, 16 to 20 degrees and 21 to 24 degrees. The occurrence of DRUJ angles based on all fifty of the Tolat et. al. cadaver specimens and representing each range of DRUJ angles by its average is shown in Table 2. Furthermore, the information of Tables 1 and 2 is summarized in the graph of FIG. 2 showing the distribution of DRUJ angles, with Type II angles being listed as negative angles and Type III angles being listed as positive angles.

### BRIEF SUMMARY OF THE INVENTION

Embodiments of the invention provide an orthopedic device comprising a stem and a head and a liner, wherein an external shape of the head is not symmetric with respect to its midplane, and wherein the head has a hole completely therethrough.

Embodiments of the invention provide an orthopedic device comprising a stern and a head and a liner, wherein the head can be assembled to the stem and the liner in a first configuration providing a first external shape and can be assembled to the stem and the liner in a second configuration providing a second external shape different from the first external shape.

Embodiments of the invention provide a kit comprising multiple ulnar heads that have different external shapes, and which can be mated with a common stem or common liner or both.

### BRIEF DESCRIPTION OF THE ILLUSTRATIONS

Embodiments of the invention are further described in the following illustrations.

FIG. 1 is a general anatomical illustration of three possible relationships between the ulna and the radius in human anatomy.

FIG. 2 is a distribution of measured Distal Radioulnar Joint (DRUJ) angles among various human patients.

FIG. 3 is a three-dimensional perspective view of an implant of the invention, in one of its configurations.

FIG. 4 is an exploded version of FIG. 3.

FIG. 5 is a partial cross-section of FIG. 3.

FIG. 6A is a three-dimensional perspective view of the stem. FIG. 6B is a cross-sectional view of FIG. 6A.

FIG. 7A is a three-dimensional perspective view of the head. FIG. 7B is a cross-sectional view of FIG. 7A.

FIG. 8A is a three-dimensional perspective view of the liner. FIG. 8B is a cross-sectional view of FIG. 8A.

FIG. 9A is a three-dimensional perspective view of the washer. FIG. 9B is a perspective cross-sectional view of the washer and a portion of the stem.

FIGS. 10A, 10B and 10C are cross-sectional views showing the ability of heads to be assembled to the rest of the implant so as to provide various different configurations in regard to the external shape presented by the head.

FIG. 11 more specifically illustrates the ability of a single head to be assembled together with other components in either of two ways, so as to provide oppositely-oriented external tangent angles.

FIG. 12A and FIG. 12B (which is a cross-section of the head depicted in FIG. 12A) illustrate an alternative design in which the head does not have a central hole therethrough but still has the ability to be assembled onto the stem in two opposite orientations.

### DETAILED DESCRIPTION OF THE INVENTION

Referring now to FIGS. 3, 4 and 5, in embodiments of the invention, there may be provided an implant 10, which may generally have a centerline 12. Centerline 12 may be an axis of rotational symmetry for at least some surfaces of at least some of the components. The implant 10 may comprise a stem 100 and a head 300 and a liner 300. Stem 100, head 200 and liner 300 may have features similar to those described in commonly assigned WO2007109752, which is hereby incorporated by reference.

Referring now to FIGS. 6A and 6B, the stem 100 may comprise a longitudinal member 110, which may in turn be followed by a widened collar 120, which may in turn be followed by a post 130. Longitudinal member 110 may be adapted to fit into the medullary canal of a long bone such as the ulna. Some features of longitudinal member 110 (such as a fiat feature in certain places an the longitudinal member 110) may be non-axisymmetric, as illustrated, or, alternatively, longitudinal member 110 may be axisymmetric. Collar 120 may be axisymmetric, and post 130 may be axisymmetric. The stem 100, such as post 130, may comprise a retention feature that is suitable to interact with a complementary feature in another component to retain the stem 100 to another component. For example, the retention feature may comprise a groove 134 that may participate in a snapping-together of certain components. For example, the groove 134 may be complementary to another feature on another component of the implant.

Referring now to FIGS. 7A and 7B, there may further be provided head 200. Head 200 may have a generally annular shape defining a hollow interior that extends from a first end 264 to a second end 266. Head 200 may be generally axisymmetric around centerline 12.

In connection with the head 200, there may be described a bounding plane such that the bounding plane is perpendicular to the centerline 12, and the bounding plane touches the head from a given direction but does not intersect the head (i.e., the head 200 exists on one side of the bounding plane and touches the bounding plane but does not exist on the opposite side of the bounding plane).

More specifically, there may be a first bounding plane 210 that non-intersectingly touches the head 200 from a first direction the head contacts the plane but only from one direction, so that the head does not extend to the opposite side of the plane). Similarly, there may be a second bounding plane 212 that non-intersectingly touches the head 200 from a second direction opposed to the first direction. The first bounding plane 210 and the second bounding plane 212 are parallel to each other. Furthermore, a midplane 214 may be defined as a plane that is halfway between the first bounding plane 210 and the second hounding plane 212. The midplane 214 may be parallel to the first bounding plane 210 and to the second bounding Wane 212.

The head 200 may have an external surface 220 that has a portion that is either cylindrical (in the case of Type I anatomy) or frustoconical (in the case of Type II or Type III geometry). It is also possible that such surface 220 may be very gently curved. External surface 220 may define a tangent line 230. Tangent line 230 may coincide with a portion of the surface in the cylindrical or frustoconical situation, or more generally, tangent line 230 may be tangent to the external surface of the head 200 at the midplane of head 200. The angle between tangent line 230 and centerline 12 may match or approximately match the Distal Radioulnar Joint angle as discussed elsewhere herein. Generally, head 200 may have curved fillets to blend between adjacent geometric portions.

With respect to the midplane 214, the head 200 may be either symmetric or non-symmetric. More specifically, the external shape of the head 200 may be either symmetric or non-symmetric with respect to the midplane 214. If the had 200 is symmetric with respect to the midplane 214, such head 200 may be appropriate to be used for a Type I ulnar geometry. If the head 200 is non-symmetric with respect to the midplane 214, such head 200 may be appropriate to be used for a Type II or a Type III ulnar geometry.

The head 200 may comprise a retention feature that is suitable to interact with a complementary feature in another component to retain the head 200 to another component. For example, in its interior the head 200 may comprise a retention lip 240 that protrudes inwardly more than most of the rest of the internal surface of head 200. As illustrated in FIG. 7B, there may be provided two such retention lips 240, one at first end 264 of head 200 and the other at second end 260 of head 200. The retention lip 240 at first end 264 may be identical to or symmetric with the retention lip 240 at second end 266. Retention lip(s) 240 may be dimensioned to be complementary with a corresponding feature on liner 300. The interior surface of head 200 may be cylindrical at places other than the retention lips 240. Other locations of retention lip(s) 240 on head 200 are also possible.

The head 200, together with other parts described herein, may be such that the head 200 can be assembled to other parts of the implant 10 in either of two configurations for a given set of parts. For a given set of parts, one configuration can provide a slope of the external surface of head 200 in one direction, with respect to centerline 12, and the other configuration can provide a slope of the external surface of head 200 in the opposite direction.

Referring now to FIGS. 8A and 8B, embodiments of the invention may also comprise a liner 300. The liner 300 may be generally annular having a hole therethrough from a first end to a second end. The liner 300 may be generally axisymmetric. The liner 300 may comprise a locking feature that under certain situations interlocks with a complementary feature of the stem 100. For example, the liner 300 may comprise inward protrusion 334 that may be complementary to groove 134 of post 130 of stem 100. On its outer surface, the liner 300 may have a depression 336 that is opposed to inward protrusion 334. The liner 300 may comprise a locking feature that under certain situations interlocks with a complementary feature of the head 200. For example, liner 300 may comprise external bulges 324 that may be complementary to retention lips 240 of head 200. The liner 300 may be sufficiently flexible so that it can be snapped into the interior of head 200 and can be snapped onto the exterior of post 130 of stem 100. This may create a non-adhesive physical interference between post 130 of stem 100, and liner 300, and head 200.

Referring now to FIG. 9A, washer 400 may be provided. Washer 400 may have a central hole through which post 130 may pass, and may be dimensioned to fit between collar 120 and head 200. With reference now to FIG. 9B, stem 100 may comprise a depression 138 that is dimensioned to cooperate with washer 400 to determine a location of washer 400 with respect to stem 100, and also to capture and retain washer 400 in its desired location. Such location may be adjacent to collar 120, in contact with collar 120. It is possible that there may be a clearance gap, as illustrated, which may be due to either design or stack-up of dimensional tolerances, between washer 400 and head 200. Washer 400 may be made of a material that is softer than the material of head 200. Washer 400 may be made of the some material as liner 300. It is possible that the softness of the material of washer 400 may help to cushion certain shock loads or other forms of load that the assembly may experience, such as during implantation.

The dimensions of various features of the stem 100, the head 200 and the liner 300 may be such that, assuming there has been pre-assembly of head 200 with liner 300, the liner 300 may be slid over the post 130 of stem 100 during a final assembly process in such a way that there is only a relatively small amount of tensile stress created in the head 200 during the process of sliding liner 300 over post 130. For example, such assembly might require only localized bending in liner 300 near the midplane of liner 300. More specifically, inward protrusion 334 may deflect radially outward, and the corresponding depression 330 on the outer surface of liner 330 may deflect similarly, to allow liner 300 with head 200 already engaged with liner 300) to slide into its assembled position with respect to post 130. Liner 300 may have a midplane that is defined similarly to the midplane 214 of head 200, and which in the assembled configuration may coincide with midplane 214 of head 200. Such bending could temporarily urge regions of liner 130 into empty interior localized space (depression 336) between liner 300 and head 200 to accomplish the snapping-in, and this bending could be relieved when the radially inward bulge 334 of liner 300 cooperates with groove 134 in post 130 of stern 100. Similarly, the dimensions of various features of the head 200 and the liner 300 may be such that, during a process of assembling head 200 with liner 300, there is only a relatively small amount of tensile stress created in the head 200 during that process. During this process, external bulges 324 of liner 300 may be compressed inward as needed to avow liner 300 to pass into its engaged position with respect to head 200. In regard to the process of sliding or snapping liner 300 into the interior of head 200, it is possible that that process could be performed while the exterior of head 200 is restrained or even compressed, so as to avoid or minimize tensile stresses in head 200 during the snapping. All of these considerations can be useful in minimizing risk to the integrity of the head 200, because in some embodiments head 200 may comprise a material that is brittle. It can be understood that a small magnitude of tensile stress may be created in head 200 during snapping-together of various components, and an even smaller magnitude of tensile stress may exist during frictional retention of the various components after snapping-together has been completed. However, this magnitude is tolerable and is smaller than would result from a assembly based on wedges or frustoconical shapes such as a Morse locking taper such as is described in U.S. Pat. No. 6,997,958. Related designs of possible engagement features are illustrated in commonly assigned U.S. Pat. Nos. 8,034,116 and 8,366,780, which are incorporated herein by reference.

According to the design of liner 300 illustrated in FIGS. 8A and 8B, a head 200 that has been assembled together with liner 300 can still be assembled to stem 100 in either of two different orientations, as shown in FIGS. 10A and 10B. This allows a single head such as the head 200 of FIGS. 10A and 10B, to accommodate both Type II and Type III anatomy, as shown in FIG. 11, with no increase in inventory.

It is possible that rounded corners may be provided in certain appropriate places on stem 100, head 200 and liner 300, and sharper corners may be provided in other appropriate places, so that snapping-together at the various parts can be performed relatively easily, but disassembly is more difficult. It is even possible that such features could be designed so that disassembly is impossible unless one or more of the parts is destroyed.

It is further possible that the dimensions of various features of the stem 100, the head 200 and the liner 300 may be such that when all three of them are assembled, there is friction among the various components so that the head 200 does not rotate freely with respect to either liner 300 or stem 100. It is possible that the relevant features of stem 100, the head 200 and the liner 300 might all be axisymmetric so that there is no preferred angular orientation and in such a situation rotation would be possible if dimensions permitted. However, in the present design it is possible that there could be sufficient friction to resist rotation, east for the conditions likely to be experienced during the activities of a patient who has received an implant.

In an embodiment of the invention, there may be provided a kit that comprises a stern 100, a head 200 and a liner 300, wherein the head 200 is annular and has a hole therethrough as described elsewhere herein and permits assembly in two different configurations for a given set of parts. The head 200 may be non-symmetric with respect to a midplane 214, as described elsewhere herein. The kit may further include another head 200 that is symmetric with respect to the midplane 214. Such a kit may provide the surgeon with a lame number of configuration choices for a given number of parts. Such elements and assemblies are illustrated in FIGS. 10A, 10B and 10C. The head 200 that is illustrated in FIG. 10B may be the same head 200 illustrated in FIG. 10A, except that it is installed m the reverse position. Such a kit may also comprise parts in various different overall sizes (e.g., small, medium and large). It is not necessary that components from one size range be assemblable with components from any other size range.

The head 200 may have, on its external surface 220, a material that may be selected for its properties of biocompatibility and resistance to wear. Such a material may be in the form of a coating that covers a substrate material making up the bulk of head 200. As an example, the coating material may be pyrolytic carbon (pyrocarbon) and the substrate material may be graphite. Graphite and pyrolytic carbon both are brittle materials, and as such are only able to tolerate a limited amount of tensile or bending stress before breaking. This stress limit can be taken into account in the design of the implant such as in features relating to the snapping-together action described elsewhere herein.

It has been found that pyrolytic carbon-coated, graphite substrates can be used to create prostheses having a modulus of elasticity that is within about 150% of the modulus of elasticity of natural bone; thus, this is considered to be an advantageous material for manufacturing such prostheses. A particular pyrocarbon is marketed as On-X® carbon (On-X Life Technologies, Inc. Austin, Tex.) (see U.S. Pat. Nos. 5,641,324 and 5,262,104). Pyrocarbon has advantageous properties for use in orthopedic prostheses such as are described herein, particularly when such is coated upon a substrate of istropic, fine grain graphite. The result is the creation of a strong prostheses that has excellent biomechanical properties. Because pyrocarbon is both physiologically inert and biochemically compatible with bone, and because the elastic modulus of such a pyrocarbon-coated graphite substrate is very close to that of cortical bone, such a prosthesis is highly biomechanically compatible and may be effectively used in such orthopedic implants, particularly those at joints within the human body where its articular surface is important. In addition to its highly compatible modulus of elasticity, pyrocarbon, provides excellent wear characteristics at its interface with bone and also with cartilage, resulting in an implant which is highly bone compatible. This is discussed in “Wear characteristics of the canine acetabulum against different femoral prostheses, ” by Stephen D. Cook, Kevin A. Thomas, Marcus A. Kester (J. Bone Joint Surg [Br] 1989; 71-B:189-97) vol. 71-B, No. 2, March 1989.

The liner 300 may be made of UHMWPE (ultra high molecular weight polyethylene). Such a material is biocompatible and has softness appropriate to allow snapping-together of the described parts without imposing a large tensile or bending stress in the head 200, which, as described elsewhere herein, might be brittle and might have limitations on the allowable tensile or bending stress. Of course, other polymeric materials and other materials generally are also possible. Washer 400 also may be made of UHMWPE. Washer 400 could be made of either the same material as liner 300 or a different material.

The stem 100 may comprise a biocompatible metal such as titanium or a titanium alloy such as is known in the art. Other materials are also possible. In general, any of the components described herein may be made of any biocompatible material having desired mechanical properties. Categories of materials for such components include metals, ceramics, polymers, and various forms of carbon as described herein or as known in the art.

It is possible to create a kit that contains heads 200 representing more than one external angle. For example, a kit may comprise one head 200 that has an external surface that is essentially parallel to the centerline 12, and it may further contain one or more heads 200 that have external surfaces that are not parallel to the centerline 12. Such heads 200 may be mountable in two opposite orientations as described elsewhere herein in order to address both Type II and Type III wrists. The number of such heads 200 provided in a kit may be determined by how finely one wishes to match external slope to a patient's anatomy. It is also possible that a kit might contain no head 200 that has its external surface exactly parallel to the centerline 12, but might contain a head 200 whose external surface angle is close to being parallel to the centerline 12, and might further contain other head(s) whose external surface has larger angle with respect to the centerline 12. Of course, a kit also might contain the described collection of head angles in several different dimensional sizes to address various overall body sizes of patients. It would be optional as to whether different dimensional sizes of heads 200 would be designed to require different dimensional sizes of other parts (stem 100, liner 300).

For the selection of a kit of drier head replacement devices having a invertible head 200 as described herein, one possible set of parts would be parts that provide four separate DRUJ angles so as to allow for treating essentially all wrist anatomies. A head portion with a DRUJ angle of 0 degrees would be appropriate for approximately 55% of patients (i.e., all of Type I wrists), a head portion with a DRUJ angle of 12.5 degrees would be appropriate for approximately 20% of patients. (81% of Type II and 2% of Type III wrists), a head portion with a DRUJ angle of 18 degrees would be appropriate for approximately 10% of patients (6% of Type II and 67% of Type III wrists) and a head portion with a DRUJ angle of 22.5 degrees would be appropriate for approximately 6% of patients (13% of Type II and 17% of Type II wrists). Further addressing issues of overall dimensions, an implant system with four DRUJ angles as just described and with three different head diameters would result in a total of 12 head components.

From a practical point of view, providing four differently-angled heads, for a given size scale or dimension, in a DRUJ kit might result in an undesirably large kit. As an alternative, it would be possible to produce a kit containing parts that provide three DRUJ angles, such as 4 degrees, 10 degrees and 16 degrees. The choice of 4 degrees, 10 degrees and 16 degrees was somewhat arbitrary and would be intended to cover a broad range of the patients represented in Table 2 and to provide prototype device that can be handled and observed to appreciate what the DRUJ angle variations look like in actual form. It should be noted that a DRUJ angle of 4 degrees was chosen to represent the Type I joint anticipating that there is some actual variation about the zero degree Type I angle. In such a situation there would not be provided a head whose external surface angle is exactly zero degrees or whose external surface is exactly cylindrical.

With use of the designs presented herein, a head 200 may be mated with the liner 300 and the stern 100 in a first configuration and in a second configuration, with the two configurations providing different external surface angle characteristics.

Alternatively, as illustrated in FIGS. 12A and 128, it is possible that head 200 could have a first blind hole 262A entering from a first end 264 of head 200 and a second blind hole 262B entering from a second end 266 of head 200. In this situation, post 130 may be short enough to fit within the respective blind holes 262A, 262B. First and second blind holes 262A and 262B may be coaxial with each other and may have equal inside dimensions and features including snap-together features such as retention lip 240.

All patents, patent applications and publications referred to herein are incorporated by reference in their entirety. The various features disclosed herein can be combined in any combination. Although the invention has been disclosed with various embodiments, the invention is not limited thereby, and is to be limited only the scope of the appended claims.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The indefinite, articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases end disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements): etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives “one or the other but not both”) when preceded by terms of exclusivity, such as “either,” “one of,” “only one of,” or “exactly one of,” “Consisting essentially, ” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended. i.e., to mean including but not limited to.

We claim:

1. An orthopedic device comprising:
a stem for engaging a bone;
a first head having a having a longitudinal centerline and having a first head external shape; and
wherein said first head can be assembled to said stem in a first configuration providing a first device external shape and wherein said first head can be assembled to said stem in a second configuration providing a second device external shape different from said first device external shape.

2. The orthopedic device of claim 1, wherein said head comprises a substrate material and a coating upon an external surface of said substrate material.

3. The orthopedic device of claim 2, wherein said substrate comprises graphite.

4. The orthopedic device of claim 2, wherein said coating composes pyrolytic carbon.

5. A kit comprising the orthopedic device of claim 1, and further comprising a second head, said second head being assemblable to said stem, said second head having a second external shape that is different from said first external shape of said first head.

6. The orthopedic device of claim 1, wherein said first head and said stern are assemblable to each other by snapping together to form said orthopedic device.

7. The orthopedic device of claim 1, wherein said stem and said head can be snapped together using less force than is required to disassemble said stem and said liner and said had from each other, or wherein said stem and said head cannot be nondestructively disassembled after they are assembled to each other.

8. The orthopedic device of claim 1, wherein said head has a hole therethrough.

9. The orthopedic device of claim 1, wherein said head comprises a first blind hole from a first end and a second blind bole from a second end.

10. The orthopedic device of claim 1, further comprising a liner that fits between said stem and said first head.

11. The orthopedic device of claim 10, wherein said liner is softer than said head and softer than said stem.

12. An orthopedic device comprising:
a stem for engaging a bone;
to first head having a longitudinal centerline and having a first head external shape; and
wherein a first bounding plane is defined as a plane perpendicular to said longitudinal centerline that non-intersectingly touches said first head from a first direction, and a second bounding plane is defined as a plane perpendicular to said longitudinal centerline that non-intersectingly touches said first head from a second direction opposed to said first direction, and wherein a midplane is defined as a plane that is midway between said first bounding plane and said second bounding plane while being parallel to said first bounding plane and said second bounding plane, and
wherein an external shape of said first head is not symmetric with respect to said midplane, and
wherein said first head has a hole completely therethrough.

13. The orthopedic device of claim 12, wherein an internal shape of said head is symmetric around said midplane.

14. The orthopedic device of claim 12, wherein said head is axisymmetric about an axis that is perpendicular to said midplane.

15. The orthopedic device of claim 12, further comprising a liner that fits between said stern and said first head.
